(19) United States
(12) Patent Application Publication
(10) Pub. No.: US 2020/0289374 A1
(43) Pub. Date: Sep. 17, 2020

(54) MICRODISPENSING WEARABLE DEVICE

(71) Applicant: Xinova, LLC, Seattle, WA (US)

(72) Inventors: Michael Keoni MANION, Seattle, WA (US); Alicia COHN, Shoreline, WA (US)

(73) Assignee: Xinova, LLC, Seattle, WA (US)

(21) Appl. No.: 16/756,522

(22) PCT Filed: Oct. 19, 2018
(86) PCT No.: PCT/US2018/056602

§ 371 Date: Apr. 16, 2020

Related U.S. Application Data
(60) Provisional application No. 62/577,119, filed on Oct. 25, 2017.

(51) Int. Cl. A61J 7/04; G16H 20/13; G16H 40/67; A61B 5/00; A61B 5/145; A61B 5/02; A61B 5/01

(52) U.S. Cl. CPC A61J 7/0409; G16H 20/13; G16H 40/67; A61B 5/6822; A61B 5/14532; A01K 27/001; A61B 5/01; A61B 5/746; A61B 2562/0271; A61B 2560/0214; A61B 2503/40; A61B 5/02

(57) ABSTRACT

A system, device, and method for dispensing a topical medication to an animal are described. The system includes a computing device and a collar. The computing device includes information related to a treatment program for dispensing a topical medication. The collar includes a container configured to store a topical medication, a microdispenser positioned on an inside surface of the collar and fluidly connected to the container, wherein the microdispenser is configured to deliver bursts of the topical medication over a period of time, and a controller operably connected to the microdispenser.

### BACKGROUND

Spot-on flea and/or tick medication for animals such as dogs and cats is effective for transdermal delivery of flea and tick deterrents. Typically, the medication is packaged in a single-use applicator. A seal on the applicator is broken or otherwise ruptured, and the medication is delivered as a single macro-dosage to the animal. The medication is then typically reapplied on a regular basis, for example, monthly or quarterly.

However, problems exist with compliance and effective application of spot-on medication, especially for animals with thick coats. Additionally, such a dosage regimen is not ideal for the treatment of fleas. Generally, the animal is overdosed immediately following the application of the medication, and underdosed towards the end of the medication cycle.

Collars exist which release medication from embedded polymer compositions integrated within the collar. However, these types of collars are wasteful as they require a whole new collar to be purchased and worn by the animal for each treatment cycle (e.g., monthly or quarterly). Additionally, such collars can be toxic to other animals that can chew on the collar, or to small children who might touch the collar and then unknowingly ingest the toxin which may have been transferred from the collar to their fingers.

### SUMMARY

A wearable device is described. In some examples, the wearable device may include a container configured to store a topical medication; a microdispenser positioned on an inside surface of the wearable device and fluidly connected to the container, where the microdispenser is configured to deliver bursts of the topical medication over a period of time; and a controller operably connected to the microdispenser and configured to provide instructions to the microdispenser to deliver the topical medication according to a treatment program. The wearable device may include, but is not limited to, a collar, a necklace, a hair clip, an armlet, a bracelet, a hair pin, an anklet, or a body chain.

A collar for dispensing a topical medication is described. In certain implementations, the collar includes: a container configured to store a topical medication; a microdispenser positioned on an inside surface of the collar and fluidly connected to the container, wherein the microdispenser is configured to deliver bursts of the topical medication over a period of time; and a controller operably connected to the microdispenser and configured to provide instructions to the microdispenser to deliver the topical medication according to a treatment program.

A method of dispensing a topical medication to an animal is also described. In certain implementations, the method includes: determining, by a controller integrated into a collar worn by the animal, timing for dispensing a topical medication based upon a treatment schedule; sending, from the controller to a microdispenser, instructions for the microdispenser to deliver at least one burst of the topical medication, wherein the microdispenser is positioned on an inside surface of the collar; receiving, at the microdispenser, the topical medication from a container fluidly attached to the microdispenser; and dispensing, by the microdispenser, an amount of the topical medication onto a portion of skin of the animal.

A system for dispensing a topical medication to an animal is also described. In certain implementations, the system includes a computing device including information related to a treatment program for dispensing a topical medication to an animal, and a collar. In some examples, the collar includes a container configured to store a topical medication, a microdispenser positioned on an inside surface of the collar and fluidly connected to the container, wherein the microdispenser is configured to deliver bursts of the topical medication over a period of time, and a controller operably connected to the microdispenser. In some examples, the controller is configured to establish communications with the computing device, receive the treatment program from the computing device, and provide instructions to the microdispenser to deliver the topical medication according to the treatment program.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the present disclosure. In the drawings:

FIG. 1 depicts an illustrative collar for dispensing a medication in accordance with an embodiment.

FIG. 2 depicts a detailed component level diagram for a collar in accordance with an embodiment.

FIG. 3 depicts a system of multiple devices in accordance with an embodiment.

FIG. 4 depicts an alternative design for a collar in accordance with an embodiment.

FIG. 5 depicts a flow diagram of an illustrative process for distributing a medication using a collar in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

The present disclosure is directed to a collar to be worn by an animal. For example, the animal may be a pet (such as a dog or a cat) or a larger animal such as production livestock animals, cattle, horses, goats, sheep, and other animals where the dispensing of a topical medications is beneficial. It should be noted that, as used herein, the phrase “topical medication” is used to refer to both site-specific medications (e.g., ointments or rash creams) as well as transdermal medications that may have a systemic effect (e.g., spot-on flea and/or tick medications).

The collar includes a refillable and/or replaceable reservoir filled with a spot-on medication. The collar also includes a dispensing device such as a microdispenser fluidly connected to the reservoir. The microdispenser may be configured to deliver small bursts or other similar controlled dosages or amounts of the medication to an animal wearing the collar over time, thereby increasing the efficiency of the medication. Rather than delivering a single macro-dosage at the beginning of a treatment cycle, the collar as described herein provides for micro-dosages of the medication throughout the treatment cycle. The collar as described herein also provides for complex treatment regimens, on-demand scheduling, multiple medication scheduling, and integrations of one or more sensors for responsive medication delivery (e.g., based upon biofeedback as described in additional detail below).

In certain implementations, the microdispenser may be configured to deliver the medication in small bursts with enough force to penetrate a fur layer of the animal, thereby delivering the medication directly to the skin of the animal. This bursting or jetting of the medication results in the collar as described herein being suitable for very furry animals or animals with double thick coats of fur.

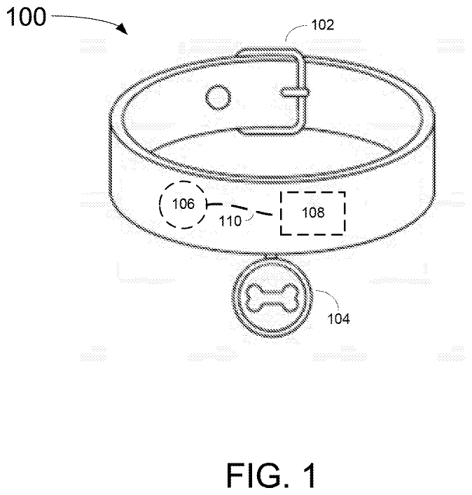

FIG. 1 illustrates a sample collar 100 incorporating the features as described herein. In some examples, the collar 100 may be made with materials similar to those used in as a standard synthetic collar. For examples, the collar 100 may be made from nylon or another similar synthetic polymer. The collar 100 may include a buckle 102 or other similar fastening device configured to allow the collar to be sized to fit various neck sizes. In certain implementations, the collar 100 may also include an identification tag 104 and/or other common collar accessories for visual decoration.

The collar 100 may also include various components for facilitating application of a topical medication to the animal wearing the collar. In certain implementations, the medication may include flea medications, insect repellants (e.g., tick repellant), pain medications, hormone medications, anti-depressant medications, vitamins, supplements, steroids, antibiotics, antiparisitics, antihistamines, antihelmintics, anti-inflammatory drugs, sedatives, tranquilizers, anxiolytics, fertility treatments, bronchodilator medications, blood pressure medications, cholesterol medications, angiotensin-converting-enzyme (ACE) inhibitors, bladder control medications, muscle relaxants, nonsteroidal anti-inflammatory drugs (NSAID), anticancer treatments, ulcer medications, decongestants, diuretics, analgesics, vasodilators, anticonvulsants, antifungal medications, cannabinoids, appetite stimulants, appetite suppressants, insecticides, and/or other similar topical medications. It should be noted that flea medication is described herein in the following discussion by way of example only, and the dispensing components and techniques as described herein may be used for application of any topical medication for application to an animal, including any of those listed above.

In certain implementations, the collar 100 may include at least one reservoir 106 configured to hold a quantity of the medication that is to be applied to the animal wearing the collar. In some examples, the reservoir 106 is sized to hold an amount of medication for a complete dosing cycle. For example, the reservoir 106 may be configured to hold enough medication for a 30-day treatment cycle. For certain spot-on flea medications, a typical dosage for a large dog (e.g. 456-88 pounds) is about 2.68 mL per month. To accommodate such an amount, the reservoir 106 may be sized appropriately. In certain implementations, the reservoir 106 may be slightly oversized to accommodate an extra amount of the medication in the event that extra dosing might be required during the treatment cycle. For example, for a large dog, the reservoir 106 may be sized to hold 3.00 mL of the flea medication. For smaller animals, the reservoir 106 may be sized to hold a lesser amount of medication. For example, the size of the reservoir 106 may range from about 1.0 mL to 2.0 mL for smaller animals, from about 2.0 mL to 3.0 mL for medium sized animals, and about 3.0 mL or greater for larger animals. In some examples, the reservoir 106 may be sized such that the amount of medication delivered to the animal is not dangerous to the animal should the reservoir rupture or otherwise fail.

The reservoir 106 may be made from a material that may withstand normal activity and is not easily punctured. For example, the reservoir 106 may be made from a thermoplastic resin using a thermo-forming process to produce a reservoir 106 that is resistant to compressing forces and twisting forces likely to be exerted on the collar 100 when worn by the animal. The reservoir 106 may also be made such that the medication contained therein is protected from dirt, water, and other debris that the collar 100 might be exposed to in normal daily use.

The collar 100 may also include a microdispenser 108. In certain implementations, the microdispenser 108 may be integrated into a microchip that is located on or within the underside of the collar 100 such that the microdispenser is positioned adjacent to the animal's skin. The microdispenser 108 may be configured to deliver a series of microdrops of medication to the animal's skin at a certain frequency for a particular period of time. For example, the microdispenser 108 may be configured to deliver a series of 50 picoliter microdrops at a frequency of about 20 Hz. The microdispenser 108 may be configured to deliver the bursts of medication with enough force to deliver the medication to the surface of the animal's skin where the medication may be absorbed, for example, by one or more hair follicles.

In certain implementations, the microdispenser 108 may be configured to deliver microdrops sized from about 25 picoliters to about 75 picoliters. In certain implementations, the microdispenser 108 may be configured to operate at a frequency of about 10 Hz to about 50 Hz.

In certain implementations, the microdispenser 108 may be a piezoelectric microdispenser configured to produce a burst or otherwise dispense an amount of liquid in response to an electrical impulse causing a piezoelectric element to eject a portion of the liquid from the microdispenser. In other implementations, the microdispenser 108 may be a thermoelectrically activated microdispenser configured to produce a burst or otherwise dispense an amount of liquid in response to a thermally reactive element increasing in temperature, causing a gas bubble to expand and eject a portion of the liquid from the microdispenser.

The reservoir 106 and the microdispenser 108 may be fluidly connected by a fluid channel 110. The fluid channel 110 may be integrated into the collar 100 itself, and be made of a flexible material that allows the fluid channel to bend with the collar. The fluid channel 110 may be attached to the reservoir 106 and the microdispenser 108 to form a watertight seal, thereby preventing dirt, water, or other debris from contaminating the medication in the reservoir or potentially clogging the microdispenser.

It should be noted that the reservoir 106, microdispenser 108, and fluid channel 110 are illustrated with dashed lines to show that the components may be positioned on the underside of the collar 100 (e.g., positioned adjacent to the animal's skin) and/or integrated into the collar itself. However, it should be noted that this placement is shown by way of example only, and the various collar components as described herein may be positioned at various places and/or positions on the collar.

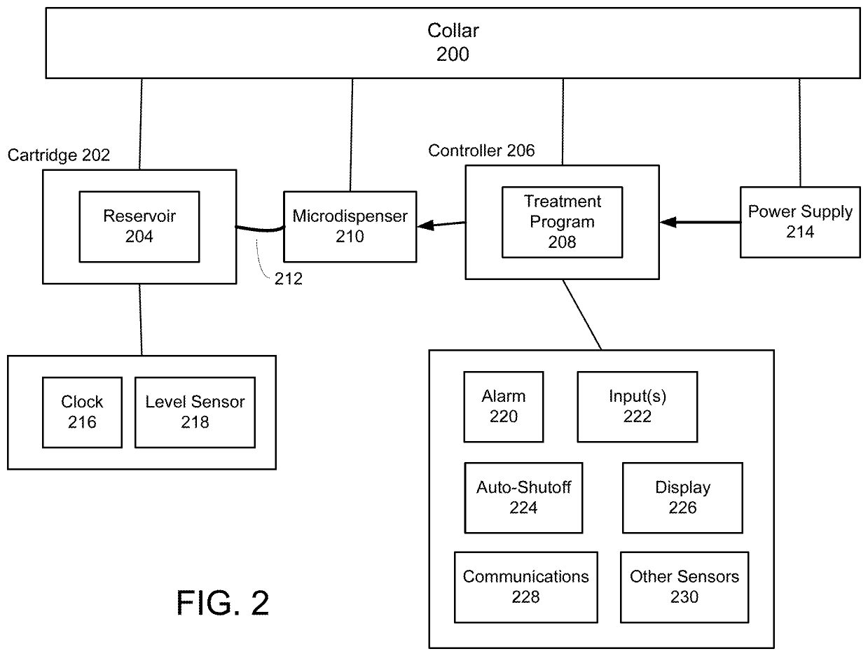

FIG. 1 illustrates the basic components included in the dispensing collar as taught and described herein. FIG. 2 illustrates a more detailed component level view of a dispensing collar including various optional features and functionality described herein.

As shown in FIG. 2, a collar 200 may include a replaceable cartridge 202. The cartridge 202 may include the reservoir 204 configured to hold a quantity of the medication to be delivered to the animal. In certain implementations, the collar 200 may be configured to quickly receive a new cartridge 202, thereby providing a full reservoir 204 including medication for another treatment cycle. However, it should be noted that including a replaceable cartridge 202 is provided by way of example only. In certain implementations, the reservoir 204 may be refillable and include, for example, a fill port or other similar opening or aperture into which additional medication may be inserted.

The collar 200 may also include a controller 206. The controller 206 may include, for example, a microprocessor or other similar processing device. The controller 206 may also include a computer-readable storage medium that is configured to store a treatment program 208. In certain implementations, the treatment program 208 may include instructions incorporating dosage and timing information for application of the medication stored, for example, in reservoir 204. Various types of treatment programs 208 may be used by the collar 200 depending upon various factors such as the animal's health, type of medication is being distributed, and other similar factors. For example, the treatment plan 208 may include a continuous release of the medication at a steady rate throughout the treatment cycle. In another example, the treatment plan 208 may include a complex release schedule, such as distributing higher dosages at the beginning of the treatment cycle and tapering the amount of medication delivered to a lower dosage at the end of the treatment cycle. The treatment program 208 may also include providing high medication dosages at random times throughout the treatment cycle to avoid continuous exposure of the animal to the high dosages. In some examples, the treatment program 208 may include periods where no medication is delivered to the animal.

The collar 200 may further include at least one microdispenser 210. The microdispenser 210 may be operably connected to the controller 206 and configured to receive dispensing instructions according to the treatment program 208. The microdispenser 210 may also be connected to the cartridge 202 via a fluid channel 212. As described above in the discussion of FIG. 1, the fluid channel 212 may be made from a flexible material and be configured to bend and move with the collar 200 as the collar is worn.

In certain implementations, the collar 200 may also include a power supply 214 configured to provide power to various components in the collar such as the controller 206 and/or the microdispenser 210. In certain implementations, the controller 206 may be configured to provide modulated power to the microdispenser 210 along with dispensing control signals. In some examples, the power supply 214 may be a rechargeable battery. For example, the power supply 214 may include a battery configured to be wirelessly charged using a standard wireless charging protocol (e.g., Qi wireless charging). In certain implementations, a charging device may be positioned near items or areas at which the animal spends a good deal of time. For example, a specialized food dish may include a wireless charger such that, when an animal wearing the collar 200 is eating or drinking, the power supply 214 may be charged. Similarly, a wireless charger may be integrated into a pet bed, blanket, or other similar object that an animal typically sleeps on or close to.

In some implementations, the power supply 214 may include kinetic recharging. In such an example, the movement of the animal wearing the collar 200 may be used to create energy that may be stored in a battery or other similar energy storage medium contained within, or operably connected to, the power supply 214.

As shown in FIG. 2, in certain implementations, various components within the collar 200 may include one or more additional features. For example, the cartridge 202 may include various additional features such as a clock 216 and a level sensor 218. The clock 216 may be an electromechanical clock that provides an indication of when the cartridge 202 is to be replaced. For example, if the treatment cycle is 30 days, the clock 216 may be a simple countdown from 30 to 0, providing a visual indication of when the cartridge 202 is due for replacement. Similarly, the level sensor 218 may also provide an indication of that status of the cartridge 202, and an indication of whether the cartridge should be replaced before the end of the treatment cycle. For example, when starting a new medication, the initial dosage levels may be higher than would normally be used throughout the treatment cycle, so the first cartridge of the new medication may be exhausted before the end of the treatment cycle. In such a scenario, the level sensor 218 may provide an indication that the medication level in the cartridge 202 is low and that the cartridge should be replaced before the end of the treatment cycle.

The controller 206 may also include various additional components and features. For example, the controller 206 may include, or be operably connected to, an alarm 220. The alarm 220 may be configured to provide an audible and/or visual indication if one or more criteria are met. For example, if the cartridge 202 has become disconnected from the collar 200, the alarm 220 may output an audible and/or visual notification that something is wrong with the collar. As described in further detail below, the alarm 220 may also be configured to operate with other features of the controller 206 to provide notification of the current status of the collar 200.

The controller 206 may further include one or more inputs 222. In certain implementations, the inputs 222 may include one or more activation/deactivation buttons configured to function as power buttons for the collar 200. In an example, the inputs 222 may include two power buttons that are spaced apart on the collar. In order to turn the collar 200 on or off, both buttons may require activation. This arrangement may prevent the animal wearing the collar 200 from accidentally turning off the power by leaning against or otherwise actuating a single button. As such, the buttons may be positioned on the collar 200 such that accidental and simultaneous activation of both buttons is unlikely. In certain implementations, the inputs 222 may further include a manual activation or dispenser control input. Such an input may provide for a person to control manual dispensing of the medication in addition to the treatment program 208. For example, if the animal has been playing outside in long grass or trees, the animal's owner may want to apply additional flea and/or tick medication immediately after the animal is done playing (or, in some examples, prior to the animal going into the grass and/or trees). In such a situation, the owner may use the manual activation control to apply additional medication.

The controller 206 may further include an auto-shutoff feature 224. The auto- shutoff feature 224 may be configured to shut off the controller 208 and the microdispenser 210 if, for example, the cartridge 202 is empty or has been removed, or if the collar has been removed from the animal.

The controller 206 may further include, or be operably connected to, a display 226. The display 226 may be configured to provide information related to the operation of the collar 200 such as status of a battery and cartridge 202 (including, for example, information received from the clock 216 and the level sensor 218), treatment program information, and the like.

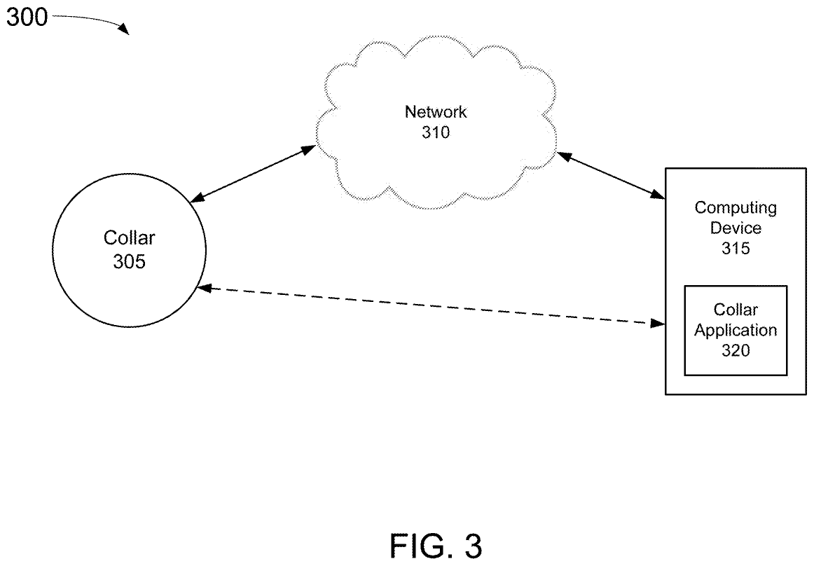

In certain implementations, the controller 206 may also include a communication interface 228. The communication interface 228 may include a wireless transceiver configured to establish a wireless communication connection with an external network and/or a remote computing device. For example, the communication interface 228 may be configured to establish a Bluetooth connection, a WI-FI connection, a mesh network connection, a near-field communication connection, or another similar wireless connection. Communications between a collar such as collar 200 and an external network and/or a remote computing device is described in additional detail in the description of FIG. 3 below.

In some examples, the controller 206 may also include other sensors 230. For example, the other sensors 230 may include a temperature sensor configured to monitor the temperature of the animal wearing the collar 200, an activity sensor configured to monitor physical activity of the animal, a heart sensor configured to monitor cardiac activity of the animal, a motion sensor configured to monitor specific movements of the animal (beyond normal activity) such as biting and/or scratching by the animal, a latch or closing sensor configured to monitor that the collar is appropriately attached to the animal, and other similar sensors. Based upon the output of the additional sensors 230, the controller 206 may be configured to alter the treatment program 208 or to otherwise alter the dispensing of the medication. For example, if the medication to be dispensed is a topical heart medication used to control an animal's heart rate and/or blood pressure, the controller 206 may use information from a heart activity sensor to determine if the dosage of the heart medication should be adjusted. For instance, if the heart activity sensor detects that the animal is exhibiting high blood pressure and/or heart rate, the medication dosage can be increased until the animal's heart activity returns to a normal level.

It should be noted that the arrangement of the components as shown in FIG. 2 is by way of example only. This is particularly true for the various additional components. For example, the clock 216 and/or the level sensor 218 may be integrated with or operably connected to the controller 206. Information related to the clock 216 (e.g., treatment cycle time remaining) or the level sensor 218 (e.g., remaining medication level) may be output on display 226 or sent to another computing device via the communication interface 228.

It should also be noted that the various additional components as described above may be optional depending upon the implementation of the collar 200. Depending upon the design, intended use, and available power for the collar 200, the amount and type of additional features included in the collar may vary accordingly.

FIG. 3 illustrates a sample system 300 for communication between a collar and a remote computing device. As described above, a collar 305 may include a communication interface configured to operably connect to a remote network such as network 310. In certain implementations, the network 310 may be implemented as a wireless network. For example, the network 310 may be a wide area network such as a WI-FI network. In other examples, the network may be a local area network such as a mesh network. A remote computing device 315 may also be configured to connect to the network 310, thereby establishing a connection with the collar 305. In certain implementations, the remote computing device 315 may be a smartphone used by the owner of an animal wearing the collar 305. In some examples, the remote computing device 315 may include an application 320 configured to interact with various features on the collar 305. For example, the application 320 may receive information from the collar 305 regarding various information related to the operation of the collar such as medication levels, dispensing history, power level, previous alarm information, and other related information. The owner may also use the application 320 to provide an updated treatment program, initiate a manual dispensing, clear alarm information, and perform additional tasks related to the operation of the collar 305.

In certain implementations, the application 320 may also be used to place the collar into a different mode or to be configured to operate using alternate settings. For example, if the owner is taking the animal into the woods for a hike, the owner may update the application 320 accordingly. The application 320 may communicate this information to the collar 305 and, based upon the updated location of the animal (e.g., the woods), the treatment program for the collar may be updated accordingly. For example, if the collar 305 is configured to dispense flea and tick medication, the collar may increase the dosage based upon this location information.

Depending upon the communication capabilities of the collar 305, a direct connection may be established between the collar and the computing device 315. For example, the collar 305 and computing device 315 may be configured to connect using Bluetooth, near field communications, or another similar short-range communication protocol.

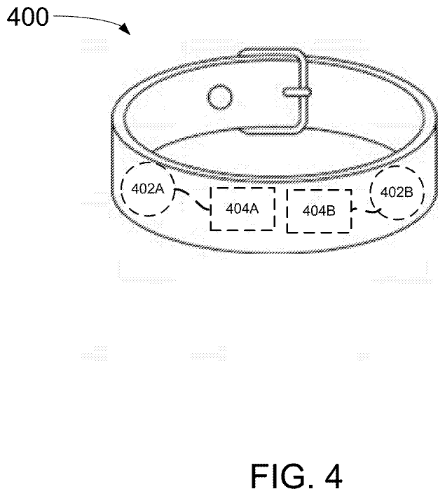

Various additional features may be included and/or integrated into a collar as described herein. For example, as illustrated in FIG. 4, a collar 400 may include multiple reservoirs 402A and 402B. Each reservoir 402A, 402B may contain a separate medication. For example, reservoir 402A may be configured to store a topical flea and tick medication, and reservoir 402B may be configured to store a vitamin and/or supplement. Each reservoir may be connected to a separate microdispenser 404A, 404B. However, it should be noted that, depending upon the design of the collar 400, a single microdispenser may be included and be fluidly connected to both reservoirs 402A and 402B.

Additionally, rather than a single microdispenser as has been described herein (or two microdispensers as shown in FIG. 4), a collar may be designed such that it includes an array of microdispensers. Such a design may increase the amount of medication that may be applied to an animal while reducing the amount of time required to apply the medication. For example, an array of 3×3 microdispensers may be incorporated into a collar design. Additionally, a collar may include additional features such as an RFID tag including owner contact information in the event that the animal becomes lost, a global positioning system tracker or a device for aiding in locating the animal if they become lost, and additional useful features related to the daily wearing of the collar.

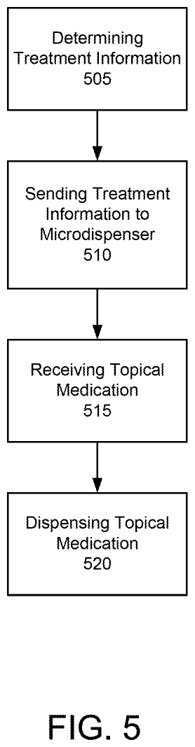

FIG. 5 illustrates a sample process for operating a collar or similar device such as those described herein to deliver a medication to an animal. A controller, such as controller 206 as described above, may determine 505 treatment information based upon the treatment schedule. For example, the controller may determine 505 timing information and dosage amount information from the treatment schedule. Based upon the treatment information, the controller may send 510 instructions to a microdispenser to deliver one or more bursts of a topical medication. The microdispenser may receive 515 an amount of the topical medication from a reservoir fluidly connected to the microdispenser. For example, the microdispenser may include an internal reservoir configured to hold a single dose, or a portion of a single dose, of the topical medication. Once the microdispenser has received 515 the topical medication, the microdispenser may dispense 520 the topical medication onto the skin of an animal wearing the collar using, as described above, a jet or burst of the topical medication.

As noted above in the discussion of FIG. 4, a collar may include multiple reservoirs and microdispensers. In such an arrangement, the process as described in FIG. 5 may be repeated for each reservoir and microdispenser pair. For example, the controller may determine 505 treatment information for dispensing a second topical medication based upon a second treatment schedule, the treatment information including timing and dosage information for the second topical medication. The controller may send 510 the instructions for the second topical medication to a second microdispenser. The second microdispenser may receive 515 the second topical medication from a second reservoir, and dispense 520 the second topical medication according to the treatment information. Depending upon the location of the second microdispenser, the second topical medication may be applied at a different location than the first topical medication.

The devices and processes as described herein provide an improved device and process for applying a topical medication to an animal using a specifically designed collar. Such an approach overcomes the drawbacks of the traditional spot-on medication techniques, primarily over-dosage at the beginning of a treatment cycle and noncompliance/improper application. However, the devices and processes as described herein may be applicable to additional uses beyond regular application of a topical medication. Specific use cases are briefly described in the following examples:

### EXAMPLE 1

An owner begins a monthly flea and tick treatment regimen. Upon initiating the regimen, the owner purchases a dispensing collar and a supply of medication cartridges. Depending upon the prescription for the animal, the size of the animal, and other factors such as health and age, the size of the collar and cartridges are chosen from a range of available sizes as would best suit that animal. The owner puts the collar on the animal, inserts the first cartridge, and turns on the collar power. The owner can also opt to install an application on their smartphone for interacting with the collar.

Upon powering on, the collar determines that the cartridge is properly installed and determines the treatment information. The treatment information can be preinstalled on the collar, can be received from the application, can be determined by the controller based upon the cartridge installed (e.g., each cartridge includes an identifier such as an RFID tag that includes treatment information), or by another similar method. The controller can then initiate the treatment regimen.

After the first treatment cycle is complete (e.g., 30 days), the collar produces an audible and/or visual alarm indicating that the cartridge needs to be replaced. If the owner has installed the application on their smartphone, the controller can send notification to the application that the cartridge is to be replaced as well. This process can repeat until the treatment regimen is completed.

### EXAMPLE 2

A owner is concerned with the amount of nutrients their pet is getting from their normal diet, but finds it difficult to give the pet a daily vitamin and/or supplements. The owner purchases a collar that includes a replaceable cartridge that can be used to provide a daily vitamin and set of supplements topically to the pet. The owner can select a generic vitamin/supplement mix, or can order a custom mix of vitamins and supplements specifically for their pet. Rather than worrying about providing the pet with a daily vitamin, the owner can merely change the cartridge monthly.

### EXAMPLE 3

A pet has been diagnosed with a heart condition and requires a daily medication to maintain their blood pressure and heart rate. The pet's owner can purchase a dispensing collar that includes integrated heart monitoring sensors. In addition to regularly dispensing the pet's medication using the processes and techniques described herein, the sensors can also monitor the pet's heart rate and blood pressure for any abnormalities. If any abnormalities are detected, the collar's controller can alter the medication's treatment program and provide notification to the pet's owner via an audible alarm and/or a notification to the owner's smartphone (e.g., via a collar specific application).

According to some examples, a wearable device may be described. The wearable device may include a container configured to store a topical medication; a microdispenser positioned on an inside surface of the wearable device and fluidly connected to the container, where the microdispenser may be configured to deliver bursts of the topical medication over a period of time; and a controller operably connected to the microdispenser and configured to provide instructions to the microdispenser to deliver the topical medication according to a treatment program.

According to other examples, the container may include a refillable reservoir. The refillable reservoir may be configured to store a quantity of the topical medication for a complete dosage cycle of the topical medication. The dosage cycle may be 7, 15, 30, 60, 90, 180, or 365 days. The refillable reservoir may include a replaceable cartridge, and the replaceable cartridge may include the topical medication. The container may include a replaceable cartridge, and the replaceable cartridge may include the topical medication. The wearable device may further include an alarm operably connected to the container and configured to emit an alarm when a level of the topical medication drops below a minimum threshold. The controller may be further configured to transmit an alert to a remote computing device when a level of the topical medication drops below a minimum threshold.

According to further examples, the microdispenser may be configured to deliver the bursts of the topical medication as a concentrated ejection configured to penetrate fur or hair of an animal wearing the wearable device to deliver the topical medication to a portion of skin of the animal. The wearable device may further include a manual control configured to cause the microdispenser to deliver an additional burst of the topical medication. The controller may be further configured to modify the treatment program based upon an activity level of an animal wearing the wearable device. The controller may be further configured to modify the treatment program based upon a location of an animal wearing the wearable device. The wearable device may further include a sensor configured to monitor a physiological state of an animal wearing the wearable device, where the controller may be further configured to modify the treatment program based upon a change in the physiological state of the animal wearing the wearable device detected by the sensor. The sensor may be temperature sensor, a blood pressure sensor, a heart rate sensor, a hormone sensor, a glucose sensor, or a biomarker sensor.

According to yet other examples, the wearable device may further include a display configured to provide an indication of when the microdispenser may be delivering a burst of the topical medication. The controller may be further configured to establish communication with a remote computing device. The controller may be further configured to receive updated treatment information from the remote computing device. The microdispenser may be configured to deliver the topical medication as a plurality of microdrops. Each of the plurality of microdrops may be about 25 picoliters to about 75 picoliters. The wearable device may further include a second container configured to store a second topical medication. The wearable device may further include a second microdispenser positioned on the inside surface of the wearable device and fluidly connected to the second container, where the second microdispenser may be configured to deliver bursts of the second topical medication over a period of time. The controller may be operably connected to the second microdispenser and configured to provide instructions to the second microdispenser to deliver the second topical medication according to a second treatment program.

According to some examples, the topical medication may be selected from the group consisting of flea medications, insect repellants, pain medications, hormone medications, anti-depressant medications, steroids, cardiac medications, appetite stimulants, appetite suppressants, antifungal medications, vitamins, and supplements. The wearable device may further include a power source. The power source may be a rechargeable battery. The rechargeable battery may include a kinetically chargeable battery. The rechargeable battery may include an induction-charged battery. The wearable device may be a collar.

According to other examples, a collar is described. The collar may include a container configured to store a topical medication; a microdispenser positioned on an inside surface of the collar and fluidly connected to the container, where the microdispenser may be configured to deliver bursts of the topical medication over a period of time; and a controller operably connected to the microdispenser and configured to provide instructions to the microdispenser to deliver the topical medication according to a treatment program.

According to further examples, the container may include a refillable reservoir. The refillable reservoir may be configured to store a quantity of the topical medication for a complete dosage cycle of the topical medication. The dosage cycle may be thirty days. The refillable reservoir may include a replaceable cartridge, wherein the replaceable cartridge may include the topical medication. The collar may further include an alarm operably connected to the container and configured to emit an alarm when a level of the topical medication drops below a minimum threshold. The microdispenser may be configured to deliver the bursts of the topical medication as a concentrated ejection configured to penetrate fur or hair of an animal wearing the collar to deliver the topical medication to a portion of skin of the animal. The collar may further include a manual control configured to cause the microdispenser to deliver an additional burst of the topical medication. The controller may be further configured to modify the treatment program based upon an activity level of an animal wearing the collar. The controller may be further configured to modify the treatment program based upon a location of an animal wearing the collar. The collar may further include a display configured to provide an indication of when the microdispenser may be delivering a burst of the topical medication. The controller may be further configured to establish communication with a remote computing device.

According to yet other examples, the controller may be further configured to receive updated treatment information from the remote computing device. The microdispenser may be configured to deliver the topical medication as a plurality of microdrops. Each of the plurality of microdrops may be about 25 picoliters to about 75 picoliters. The collar may further include a second container configured to store a second topical medication. The collar may further include a second microdispenser positioned on the inside surface of the collar and fluidly connected to the second container, and the second microdispenser may be configured to deliver bursts of the second topical medication over a period of time. The controller may be operably connected to the second microdispenser and configured to provide instructions to the second microdispenser to deliver the second topical medication according to a second treatment program. The topical medication may be selected from the group consisting of flea medications, insect repellants, pain medications, hormone medications, anti-depressant medications, steroids, cardiac medications, appetite stimulants, appetite suppressants, antifungal medications, vitamins, and supplements. The collar may further include a power source. The power source may be a rechargeable battery. The rechargeable battery may include a kinetically chargeable battery. The rechargeable battery may include an induction-charged battery.

According to further examples, a method of dispensing a topical medication to an animal is described. The method may include determining, by a controller integrated into a collar worn by the animal, timing for dispensing a topical medication based upon a treatment schedule; sending, from the controller to a microdispenser, instructions for the microdispenser to deliver at least one burst of the topical medication, where the microdispenser may be positioned on an inside surface of the collar; receiving, at the microdispenser, the topical medication from a container fluidly attached to the microdispenser; and dispensing, by the microdispenser, an amount of the topical medication onto a portion of skin of the animal.

According to some examples, the container may include a refillable reservoir configured to store a quantity of the topical medication for a complete dosage cycle of the topical medication. The method may further include emitting, by an alarm operably connected to the container, an alarm when a level of the topical medication drops below a minimum threshold. Dispensing an amount of the topical medication may include delivering, by the microdispenser, the bursts of the topical medication as a concentrated ejection configured to penetrate fur or hair of the animal wearing the collar to deliver the topical medication to the skin of the animal. The method may further include receiving, by the microdispenser, an instruction from a manual control to deliver an additional burst of the topical medication. The method may further include modifying, by the controller, the treatment program based upon an activity level of the animal. The method may further include modifying, by the controller, the treatment program based upon a location of the animal. The method may further include displaying, by a display operably connected to the controller, an indication of when the microdispenser may be delivering a burst of the topical medication.

According to other examples, the method may further include establishing, by the controller, communication with a remote computing device; and receiving, by the controller, updated treatment information from the remote computing device. The microdispenser may be configured to dispense the topical medication as a plurality of microdrops. Each of the plurality of microdrops may be about 25 picoliters to about 75 picoliters. The method may further include determining, by the controller, timing for dispensing a second topical medication based upon a second treatment schedule; sending, from the controller to a second microdispenser, instructions for the second microdispenser to deliver at least one burst of the second topical medication, where the second microdispenser may be positioned on an inside surface of the collar; receiving, at the second microdispenser, the second topical medication from a second container fluidly attached to the second microdispenser; and dispensing, by the second microdispenser, an amount of the second topical medication onto a second portion of skin of the animal. The topical medication may be selected from the group consisting of flea medications, insect repellants, pain medications, hormone medications, anti-depressant medications, steroids, cardiac medications, appetite stimulants, appetite suppressants, antifungal medications, vitamins, and supplements.

According to yet other examples, a system for dispensing a topical medication to an animal is described. The system may include a computing device may include information related to a treatment program for dispensing a topical medication to an animal and a collar. The collar may include a container configured to store a topical medication, a microdispenser positioned on an inside surface of the collar and fluidly connected to the container, where the microdispenser may be configured to deliver bursts of the topical medication over a period of time, and a controller operably connected to the microdispenser and configured to establish communications with the computing device, receive the treatment program from the computing device, and provide instructions to the microdispenser to deliver the topical medication according to the treatment program.

According to some examples, the computing device may be further configured to receive an updated treatment program from a user of the computing device; and transmit the updated treatment program to the controller. The controller may be further configured to receive the updated treatment program from the computing device and provide updated instructions to the microdispenser to deliver the topical medication according to the updated treatment program. The container may include a refillable reservoir configured to store a quantity of the topical medication for a complete dosage cycle of the topical medication. The refillable reservoir may include a replaceable cartridge, and the replaceable cartridge may include the topical medication. The collar further may include an alarm operably connected to the container and configured to emit an alarm when a level of the topical medication drops below a minimum threshold. The microdispenser may be configured to deliver the bursts of the topical medication as a concentrated ejection configured to penetrate fur or hair of an animal wearing the collar to deliver the topical medication to a portion of skin of the animal.

According to further examples, the collar may further include a manual control configured to cause the microdispenser to deliver an additional burst of the topical medication. The collar may further include a display configured to provide an indication of when the microdispenser may be delivering a burst of the topical medication. The microdispenser may be configured to deliver the topical medication as a plurality of microdrops. Each of the plurality of microdrops may be about 25 picoliters to about 75 picoliters. The collar may further include a second container configured to store a second topical medication. The collar may further include a second microdispenser positioned on the inside surface of the collar and fluidly connected to the second container, where the second microdispenser may be configured to deliver bursts of the second topical medication over a period of time. The controller may be operably connected to the second microdispenser and configured to provide instructions to the second microdispenser to deliver the second topical medication according to a second treatment program. The topical medication may be selected from the group consisting of flea medications, insect repellants, pain medications, hormone medications, anti-depressant medications, steroids, cardiac medications, appetite stimulants, appetite suppressants, antifungal medications, vitamins, and supplements. The collar may further include a power source. The power source may be a rechargeable battery.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the claims include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A wearable device to be worn by an animal, the wearable device comprising:
a container configured to store a topical medication;
a microdispenser positioned on an inside surface of the wearable device and fluidly coupled to the container, wherein the microdispenser is configured to deliver bursts of the topical medication over a period of time; and
a controller operably coupled to the microdispenser and configured to provide instructions to the microdispenser to deliver the topical medication according to a treatment program, wherein the treatment program is modified based on a location of the animal wearing the wearable device.

2. The wearable device of claim 1, wherein the container comprises a refillable reservoir.

3-6. (canceled)

7. The wearable device of claim 1, further comprising an alarm operably connected to the container and configured to emit an alarm or transmit an alert to a remote computing device when a level of the topical medication drops below a minimum threshold.

8. (canceled)

9. The wearable device of claim 1, wherein the microdispenser is configured to deliver the bursts of the topical medication as a concentrated ejection configured to penetrate fur or hair of the animal wearing the wearable device to deliver the topical medication to a portion of skin of the animal.

10. (canceled)

11. The wearable device of claim 1, wherein the controller is further configured to modify the treatment program based upon an activity level of the animal wearing the wearable device.

12. (canceled)

13. The wearable device of claim 1, further comprising a sensor configured to monitor a physiological state of the animal wearing the wearable device, wherein the controller is further configured to modify the treatment program based upon a change in the physiological state of the animal wearing the wearable device detected by the sensor and the sensor includes a temperature sensor, a blood pressure sensor, a heart rate sensor, a hormone sensor, a glucose sensor, or a biomarker sensor.

14-19. (canceled)

20. The wearable device of claim 1, further comprising a second container configured to store a second topical medication and a second fluidly coupled to the second container, wherein the controller is operably coupled to the second microdispenser and configured to provide instructions to the second microdispenser to deliver the second topical medication according to a second treatment program.

21-22. (canceled)

23. The wearable device of claim 1, wherein the topical medication is selected from the group consisting of flea medications, insect repellants, pain medications, hormone medications, anti-depressant medications, steroids, cardiac medications, appetite stimulants, appetite suppressants, antifungal medications, vitamins, and supplements.

24-28. (canceled)

29. A collar to be worn by an animal, the collar comprising:
a container configured to store a topical medication;
a microdispenser positioned on an inside surface of the collar and fluidly coupled to the container, wherein the microdispenser is configured to deliver bursts of the topical medication over a period of time; and
a controller operably coupled to the microdispenser and configured to provide instructions to the microdispenser to deliver the topical medication according to a treatment program, wherein the treatment program is modified based on a location of the animal wearing the collar.

30-32. (canceled)

33. The collar of claim 29, wherein the container comprises a replaceable cartridge configured to store the topical medication.

34-38. (canceled)

39. The collar of claim 29, further comprising a display configured to provide an indication of when the microdispenser is delivering a burst of the topical medication.

40. The collar of claim 29, wherein the controller is further configured to establish communication with a remote computing device and receive updated treatment information from the remote computing device.

41-43. (canceled)

44. The collar of claim 29, further comprising a second container configured to store a second topical medication and a second microdispenser positioned on the inside surface of the collar and fluidly coupled to the second container, wherein the second microdispenser is configured to deliver bursts of the second topical medication over a period of time.

45-47. (canceled)

48. The collar of claim 29, further comprising a power source that includes a kinetically chargeable battery or an induction-charged battery.

49-51. (canceled)

52. A method of dispensing a topical medication to an animal, the method comprising:
determining, by a controller integrated into a collar worn by the animal, a timing for dispensing a topical medication based upon a treatment schedule and one or more of a dosage or a type of the topical medication to be delivered based upon a location of the animal wearing the collar;
sending, from the controller to a microdispenser, instructions for the microdispenser to deliver at least one burst of the topical medication, wherein the microdispenser is positioned on an inside surface of the collar;
receiving, at the microdispenser, the topical medication from a container fluidly attached to the microdispenser; and
dispensing, by the microdispenser, an amount of the topical medication onto a portion of skin of the animal.

53. (canceled)

54. The method of claim 52, further comprising emitting, by an alarm device operably coupled to the container, an alarm when a level of the topical medication drops below a minimum threshold.

55. The method of claim 52, wherein dispensing an amount of the topical medication comprises delivering, by the microdispenser, the bursts of the topical medication as a concentrated ejection configured to penetrate fur or hair of the animal wearing the collar to deliver the topical medication to the skin of the animal.

56. The method of claim 52, further comprising receiving, by the microdispenser, an instruction from a manual control to deliver an additional burst of the topical medication.

57. The method of claim 52, further comprising modifying, by the controller, the treatment program based upon an activity level of the animal.

58-62. (canceled)

63. The method of claim 52, further comprising:
determining, by the controller, timing for dispensing a second topical medication based upon a second treatment schedule;
sending, from the controller to a second microdispenser, instructions for the second microdispenser to deliver at least one burst of the second topical medication, wherein the second microdispenser is positioned on an inside surface of the collar;
receiving, at the second microdispenser, the second topical medication from a second container fluidly attached to the second microdispenser; and
dispensing, by the second microdispenser, an amount of the second topical medication onto a second portion of skin of the animal.

64-81. (canceled)
